(19) United States
(12) Patent Application Publication
(10) Pub. No.: US 2009/0004280 A1
(43) Pub. Date: Jan. 1, 2009

(54) KIT FOR CARING FOR THE SKIN INTENDED TO SOFTEN CUTANEOUS SIGNS OF AGEING

(75) Inventor: Catherine Marion, Antony (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 12/208,484

(22) Filed: Sep. 11, 2008

Related U.S. Application Data
(62) Division of application No. 11/287,420, filed on Nov. 28, 2005.
(60) Provisional application No. 60/663,202, filed on Mar. 21, 2005.

(30) Foreign Application Priority Data
Dec. 17, 2004 (FR) 0453043

(51) Int. Cl. A61K 9/14; A61K 33/08; A61K 31/375; A61K 8/26; A61Q 17/04

(52) U.S. Cl. 424/489; 424/691; 424/692; 514/474; 424/59

(57) ABSTRACT

A kit for skin care intended to soften cutaneous signs of ageing, contains in separate packagings, a microdermabrasion composition, a peeling composition, a soothing composition and an anti-ageing composition.

### CROSS REFERENCE TO RELATED APPLICATIONS

This application is a divisional application of prior U.S. patent application Ser. No. 11/287,420, filed Nov. 28, 2005. The parent application claims priority to U.S. provisional application No. 60/663,202 filed Mar. 21, 2005, and to French patent application 0453043 filed Dec. 17, 2004. The disclosures of the parent and both priority documents are all incorporated herein by reference.

### BACKGROUND OF THE INVENTION

1. Field of the Invention

The present invention relates to a method for caring for the skin, preferably intended to soften cutaneous signs of ageing, comprising the successive application to the skin, preferably wetted beforehand, of four compositions, namely: a microdermabrasion composition, a peeling composition, a soothing composition and an anti-ageing composition having a given constitution.

It also relates to a kit comprising, for example in four separate containers or areas, each of the abovementioned compositions.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

2. Description of the Background

Women, indeed even men, currently have a tendency to wish to appear young for as long as possible and they are consequently seeking to tone down signs of the ageing of the skin, which are reflected in particular by wrinkles and fine lines.

To date, wrinkles and fine lines were treated using cosmetic products comprising active principles which act either at the surface of the skin or in the dermis.

Provision has thus been made to use active principles having an effect on the replacement of epidermal cells and/or a keratolytic effect, which active principles are present in leave-on compositions (generally in the form of hydroxy acids) or in peeling compositions intended to be used by the subject herself or in the salon of a skin specialist (depending on the depth of the peeling). In addition to these “chemical” desquamating agents, the use is known of abrasives, such as aluminium oxide, for exfoliating the surface of the skin. This exfoliation process is known under the term of “microdermabrasion”. Microdermabrasion is employed in particular by beauticians or skin specialists who have recourse to aluminium oxide microcrystals for resurfacing the surface layers of the skin. These crystals are sprayed over the skin using a device which subsequently sucks them up with the abraded skin.

It is known to contribute to the skin, among active principles having a dermal effect, active principles which promote the synthesis, or which prevent the deterioration, of elastic fibres (collagen and elastin) or of glycosaminoglycans of which cutaneous tissue is composed.

It is not thereby less the case that the need remains to have available a method which makes it possible to effectively combat wrinkles, which has effects which are rapidly visible and which has a long-term action.

### SUMMARY OF THE INVENTION

In point of fact, the inventor has discovered that it is possible to satisfy this need by following a protocol employing four compositions applied successively.

A subject-matter of the present invention is thus a method for caring for the skin, preferably one that is used by those intending to soften cutaneous signs of ageing, comprising the successive application, to an area of skin that has preferably been wetted beforehand:
of a microdermabrasion composition comprising at least 5% by weight of metal oxide particles, which is applied by massaging, preferably for at least one minute, and then removed by washing,
of a peeling composition comprising at least 3% by weight of at least one desquamating agent,
of a soothing composition comprising at least one of a thermal or mineral water having a mineral content of at least 300 mg/l,
of an anti-ageing composition comprising at least one anti-ageing active principle chosen from: compounds which enhance the synthesis of collagen and/or of elastin and/or of glycosaminoglycans and/or of proteoglycans and/or of fibronectin and/or of laminin; compounds which inhibit the decomposition of collagen and/or of elastin; skin relaxants; agents which inhibit the glycation of proteins; agents which enhance the proliferation of keratinocytes and/or of fibroblasts; agents which enhance the differentiation of keratinocytes; and their mixtures.

Preferably, the area of skin on which the above method is carried out is an area of skin of the face, preferably the entire face, except perhaps for the outline of the eyes.

### DETAILED DESCRIPTION OF THE PREFERRED EMBODIMENTS

The first stage of the method according to the invention, hereinafter denoted by microderm abrasion stage, is a type of exfoliation intended to resurface the surface layers of the skin. This stage comprises applying, to the skin, a composition comprising at least 5% by weight of metal oxide particles, which can, for example, be coated, in particular with a silicone compound, but are preferably uncoated.

It is advantageously a magnesium oxide or an aluminium oxide. These particles advantageously exhibit a purity of at least 95%, better still of at least 99%. Their mean particle size preferably ranges from 100 to 180 μm. Aluminium oxide is preferred for use in the present invention, in particular in the anhydrous crystalline form (corundum).

For example, use may be made, as aluminium oxide particles, of particles calcined at high temperature, until the crystalline structure of corundum α-Al₂O₃) is obtained, and then treated in order to form grains having sharp edges and having a given particle size distribution, the particles preferably having a mean particle diameter of between 100 and 180 μm and more preferably between 130 and 150 μm. Their distribution is advantageously such that none of the particles has a diameter of greater than 250 μm. Such particles are available commercially in particular from MarkeTech International under the trade name Dermagrain. The particles referenced Dermagrain 900 are composed of crystalline α-alumina with a purity of 99.55% exhibiting a mean particle diameter of approximately 140 μm, the particles all having a diameter of less than 250 μm. Less than 3% of the particles have a diameter of less than 105 μm. Other particles are available from Industrial Supply under the trade names ARL 100 and ARL 120. They are aluminium oxide particles having a mean particle size of 120 and 100 μm respectively and a particle size distribution ranging from 75 to 212 μm and from 63 to 180 μm respectively.

In an alternative form, the metal oxide used in the composition according to the invention can be magnesium oxide preferably having a mean particle size ranging from 100 to 180 μm. An example of particles of this type is sold by MarkeTech International under the trade name Magnaderm 100. They are particles having a mean particle diameter of approximately 120 μm and a purity of at least 99%.

The metal oxide can represent, for example, from 5 to 40%, preferably from 10 to 30% and more preferably 20% of the total weight of the composition employed in the first stage of the method according to the invention. This composition preferably has a pH of greater than 4 and less than 8, better still less or equal to 7 and even better still of between 5.5 and 7.

The composition used in the first stage of the process according to the invention can be provided in any form, including in the form of a lotion, gel, fluid or cream. It can comprise various adjuvants and advantageously includes at least one heterogeneous polysaccharide. This is because these compounds make it possible to reduce the discomfort associated with the use of the abovementioned abrasive metal oxide particles. This heterogeneous polysaccharide can be an alginate or alternatively a heterogeneous polysaccharide comprising at least one fucose unit, comprising in particular fucose, galactose and galacturonic acid units, in particular a linear sequence of α-L-fucose, of α-D-galactose and of galacturonic acid. Such a polysaccharide is available in particular in the form of a 1% solution in water from Solabia under the trade name Fucogel 1000 PP®.

This composition can be applied in any manner, for example by spraying the composition over the skin using a device which subsequently sucks it up with the abraded skin. In an alternative form, this composition can be applied to the skin by manual massaging with the fingertips or by mechanical massaging using a vibrating device provided with a massaging head equipped with a pad, as disclosed in Application US 2001/0046506 or patent U.S. Pat. No. 6,652,888, for example. The composition is subsequently removed by washing. To do this, the skin will generally be rinsed with water.

The second stage of the process according to the invention, or peeling stage, comprises the application to the skin of a composition comprises at least 3% by weight of at least one desquamating agent.

This desquamating agent can be chosen in particular from: α-hydroxy acids, such as citric, lactic, glycolic, malic, tartaric or mandelic acid; β-hydroxy acids, such as salicylic acid or 5-(n-octanoyl)salicylic acid; urea; aminosulphonic compounds and in particular N-(2-hydroxyethyl)piperazine-N′-2-ethanesulphonic acid (HEPES); and derivatives of 2-oxothiazolidine-4-carboxylic acid (procysteine). Lactic acid, glycolic acid, HEPES and their mixtures are preferred for use in the present invention.

The amount of desquamating agent can for example represent from 3 to 15% of the weight of the composition employed in the second stage of the process according to the invention. Use may thus be made of 10% of glycolic acid or of a mixture comprising 3.5% of glycolic acid, 0.5% of lactic acid and 5% of HEPES, with respect to the total weight of the composition.

This composition preferably has a pH of between 2.5 and 6, advantageously of between 3.5 and 4.5.

It can be applied to the skin in any form including in the form of a lotion, gel, fluid or cream. In an alternative form, it can be applied in the form of a mask or patch or can be impregnated on a wipe. These implementational forms have the advantage of making possible packaging of the composition in the form of single-dose compositions, avoiding the application to the skin of an excessive amount of desquamating agent capable of producing undesirable effects. In addition, they make it possible to observe a minimum leave-on time which will depend on the nature and on the concentration of the desquamating agent, ensuring that the desired result is obtained.

In order to bring the skin to a pH close to 6 and to sooth it, the second stage of the process according to the invention is followed by the application to the skin of a composition comprising a thermal or mineral water having a mineral content of at least 300 mg/l.

In the invention, the term “mineral content” is understood to mean the sum of the concentrations of anions and of cations present in the thermal or mineral water. The fact of using a water with a high mineral content makes it possible to compensate for the irritating effect of the aluminium oxide particles and of the desquamating agents employed previously.

In the present invention, use is made without distinction of a thermal water or of a mineral water. Generally, a mineral water is suitable for consumption, which is not always the case with a thermal water. Each of these waters comprises, inter alia, trace elements and dissolved minerals.

The thermal and/or mineral water used according to the invention can have a mineral content of at least 400 mg/l, in particular of at least 700 mg/l, and more particularly a total concentration of carbonates and of bicarbonates of at least 150 mg/l and more preferably of at least 360 mg/l and in particular of sodium carbonate and bicarbonate of greater than 2 mg/l. The concentration of silicon oxide in the water used in the composition according to the invention can preferably be at least 6 mg/l and more preferably at least 9 mg/l.

The thermal water or the mineral water used according to the invention can for example be chosen from water from Avène, water from Vittel, waters from the Vichy basin, water from Uriage, water from La Roche Posay, water from La Bourboule, water from Enghien-les-Bains, water from Saint Gervais-les-Bains, water from Néris-les-Bains, water from Allevard-les-Bains, water from Digne, water from Maizières, water from Neyrac-les-Bains, water from Lons-le-Saunier, water from Eaux-Bonnes, water from Rochefort, water from Saint Christau, water from Les Fumades and water from Tercis-les-Bains.

Among these waters, those which exhibit a mineral content of less than 700 mg/l but of greater than 400 mg/l are water from La Roche Posay, water from Eaux-Bonnes or water from Saint Christau.

Among these waters, those which exhibit a total concentration of carbonates or bicarbonates of greater than 360 mg/l are water from Vittel, water from La Bourboule, water from Les Fumades, water from Enghien-les-Bains, water from La Roche Posay, water from the Vichy basin or water from Uriage.

Among these waters, those which exhibit a concentration of carbonates or bicarbonates of between 150 mg/l and 360 mg/l are water from Digne, water from Maizieres, water from Rochefort or water from Saint Gervais-les-Bains.

Among these waters, those which comprise at least 2 mg/l of sodium carbonate or bicarbonate are water from La Roche Posay, water from Vittel, waters from the Vichy basin and water from Uriage.

The waters comprising at least 9 mg/l of silicon oxide are water from La Roche Posay, water from Vittel, waters from the Vichy basin or water from Uriage.

Waters from the Vichy basin are preferred for use in the present invention.

The soothing composition employed in the third stage of the process according to the invention generally preferably comprises more than 40% by weight, preferably more than 60% by weight, more preferably more than 80% by weight, indeed even 100% by weight, of a mineral or thermal water as defined above.

After this “neutralization” stage, the fourth stage of the process according to the invention is a “maintenance” stage, comprising the application to the skin of an anti-ageing composition comprising at least one anti-ageing active principle chosen from:
compounds which enhance the synthesis of collagen (such as extracts of Centella asiatica; asiaticosides and derivatives; ascorbic acid or vitamin C and its derivatives; synthetic peptides, such as iamine, biopeptide CL or palmitoyloligopeptide, sold by Sederma; peptides extracted from plants, such as the soybean hydrolysate sold by Coletica under the trade name Phytokine®; and plant hormones, such as auxins and lignans) and/or of elastin (such as the extract of Saccharomyces cerevisiae sold by LSN under the trade name Cytovitin®; and the extract of the alga Macrocystis pyrifera sold by Secma under the trade name Kelpadelie®) and/or of glycosaminoglycans (such as the product of fermentation of milk by Lactobacillus vulgaris sold by Brooks under the trade name Biomin yogourth®; the extract of the brown alga Padina pavonica sold by Alban Müller under the trade name HSP3®; and the extract of Saccharomyces cerevisiae available in particular from Silab under the trade name Firmalift® or from LSN under the trade name Cytovitin®) and/or of proteoglycans and/or of fibronectin (such as the extract of Salina zooplankton sold by Seporga under the trade name GP4G®; the yeast extract available in particular from Alban Müller under the trade name Drieline®; and the palmitoyl pentapeptide sold by Sederma under the trade name Matrixil®) and/or of laminin;
compounds which inhibit the decomposition of collagen (such as retinoids and derivatives, oligopeptides and lipopeptides, lipoamino acids, the malt extract sold by Coletica under the trade name Collalift®; extracts of blueberry or of rosemary; lycopene; isoflavones, their derivatives or the plant extracts comprising them, in particular extracts of soybean, for example sold by Ichimaru Pharcos under the trade name Flavosterone SB®, of red clover, of flax, of kakkon or of sage) and/or of elastin (such as the peptide extract of Pisum sativum seeds sold by LSN under the trade name Parelastyl®; heparinoids; and pseudodipeptides, such as {2-[acetyl(3-(trifluoromethyl)phenyl)amino]-3-methylbutyrylamino}acetic acid);
skin relaxants, such as alverine and its salts, manganese salts and in particular manganese gluconate, magnesium salts and in particular magnesium gluconate and sulphate, the hexapeptide Argireline R, sold by Lipotec, adenosine, and also sapogenins and natural extracts, in particular ofDioscorea opposita or of Dioscorea villosa (wild yam), comprising them, and also extracts of Boswellia serrata;
agents which inhibit the glycation of proteins, such as extracts of plants from the family of the Ericaceae, in particular an extract of blueberry (Vaccinium angustifolium); ergothioneine and its derivatives; and hydroxystilbenes and their derivatives, such as resveratrol and 3,3′,5,5′-tetrahydroxystilbene;
agents which enhance the proliferation of keratinocytes (such as retinoids, including retinol and retinyl palmitate, adenosine, phloroglucinol, the extracts of walnut meal sold by Gattefossé and the extracts of Solanum tuberosum sold by Sederma) and/or of fibroblasts (such as plant proteins or polypeptides, plant extracts, in particular of soybean, and plant hormones, such as gibberellins and cytokinins);
agents which enhance the differentiation of keratinocytes, such as inorganic materials, including calcium; a lupin peptide extract, such as that sold by Silab under the trade name Structurine®; sodium β-sitosteryl sulphate, such as that sold by Seporga under the trade name Phytocohesine®; a water-soluble maize extract, such as that sold by Solabia under the trade name Phytovityl®; a peptide extract of Voandzeia subterranea, such as that sold by Laboratoires Sérobiologiques under the trade name Filladyn LS 9397®; and lignans, such as secoisolariciresinol; and their mixtures.

Among these compounds, preference is very particularly given to ascorbic acid and/or its derivatives, such as ascorbyl glucoside, adenosine and their mixtures.

The anti-ageing active principle can for example represent from 0.001 to 10%, preferably from 0.01 to 5%, of the total weight of the composition employed in the fourth stage of the process according to the invention.

This composition, and for that matter all compositions described herein, generally preferably comprise a physiologically acceptable medium, that is to say a medium compatible with the skin and/or its superficial body growths. It is preferably a cosmetically acceptable medium, that is to say one which exhibits a pleasant colour, a pleasant smell and a pleasant feel and which does not result in unacceptable discomfort (tingling, tightness, red blotches) liable to dissuade the consumer from using this composition. The pH of these compositions, which is advantageously similar to that of the skin, is generally between 5 and 7 and preferably ranges from 5.5 to 6.5 unless stated otherwise above.

The compositions employed in the various stages of the process according to the invention can be provided in all the dosage forms used for topical application and in particular in the form of dispersions of the lotion or gel type, of emulsions with a liquid or semi-liquid consistency of the milk type, obtained by dispersion of a fatty phase in an aqueous phase (O/W) or vice versa (W/O), of suspensions or emulsions with a soft, semi-solid or solid consistency of the cream or gel type, of multiple emulsions (W/O/W or O/W/O), of microemulsions, of vesicular dispersions of ionic and/or nonionic type, or of wax/aqueous phase dispersions. These compositions are prepared according to the usual methods.

According to a preferred embodiment of the invention, the microdermabrasion and/or anti-ageing compositions (respectively employed in the first and/or in the fourth stage) are provided in the form of an oil-in-water (O/W) emulsion. The oils present in these emulsions can be silicone oils, which may be volatile or nonvolatile, hydrocarbon oils or vegetable oils. These emulsions can additionally comprise non-oily fatty substances, such as shea butter, silicone gums, esters of fatty acids and of fatty alcohols, fatty acids and fatty alcohols.

In addition, the peeling composition employed in the second stage is preferably provided in the form of an aqueous gel.

Finally, the soothing composition employed in the third stage is preferably provided in a vaporizable form.

These compositions can additionally comprise various adjuvants, including for example those commonly used in the cosmetics field, such as emulsifiers, including glyceryl fatty acid esters, sugar fatty acid esters, sorbitan fatty acid esters, polyethylene glycol fatty acid esters, ethoxylated fatty alcohols and alkylpolyglycosides; fillers, in particular polyacrylamide (Nylon) fibres and/or microbeads, silica, optionally in the form of a colloidal dispersion, and/or organic microspheres which are optionally expanded; preservatives and/or copreservatives, such as caprylyl glycol; sequestering agents, such as EDTA salts; colorants; fragrances; pH adjusters, such as neutralizing agents and/or buffering agents; ethanol; and thickening and gelling agents, in particular acrylamide homo- and copolymers, acrylic homo- and copolymers, acrylamidomethylpropanesulphonic acid (AMPS) homo- and copolymers, and xanthan gum.

Of course, a person skilled in the art will take care to chose this or these possible additional compounds and/or their amounts so that the advantageous properties of the compositions according to the invention are not, or not substantially, detrimentally affected by the envisaged addition.

In addition, the composition employed in the fourth stage of the process according to the invention preferably comprises additives which make it possible to protect the skin, weakened previously, with respect to further assaults, in particular from the environment.

It therefore advantageously comprises at least one moisturizing agent, such as polyols, including glycerol, at least one antioxidant or agent for combating free radicals, such as tocopherol, tocopherol acetate, ascorbic acid and arginine pyrrolidonecarboxylate, at least one organic or inorganic photoprotective agent which is active in the UV-A and/or UV-B region, or a mixture of such agents.

The organic photoprotective agents are preferably chosen in particular from anthranilates; cinnamic derivatives; dibenzoylmethane derivatives; salicylic derivatives; camphor derivatives; triazine derivatives, such as those disclosed in patent applications U.S. Pat. No. 4,367,390, EP 863 145, EP 517 104, EP 570 838, EP 796 851, EP 775 698, EP 878 469, EP 933 376, EP 507 691, EP 507 692, EP 790 243 or EP 944 624; benzophenone derivatives; β,β-diphenylacrylate derivatives; benzotriazole derivatives; benzalmalonate derivatives; benzimidazole derivatives; imidazolines; bisbenzoazolyl derivatives, such as disclosed in Patents EP 669 323 and U.S. Pat. No. 2,463,264; p-aminobenzoic acid (PABA) derivatives; methylenebis(hydroxyphenylbenzotriazole) derivatives, such as disclosed in Applications U.S. Pat. No. 5,237,071, U.S. Pat. No. 5,166,355, GB 2 303 549, DE 197 26 184 and EP 893 119; benzoxazole derivatives, such as disclosed in Patent Applications EP 0 832 642, EP 1 027 883, EP 1 300 137 and DE 10162844; screening polymers and screening silicones, such as those disclosed in particular in Application WO 93/04665; dimers derived from α-alkylstyrene, such as those disclosed in Patent Application DE 19855649; 4,4-diarylbutadienes, such as disclosed in Applications EP 0 967 200, DE 19746654, DE 19755649, EP-A-1 008 586, EP 1 133 980 and EP 133 981, and their mixtures.

The organic photoprotective agents more particularly preferred are chosen from the following compounds (CTFA names or chemical names):
Ethylhexyl Salicylate,
Ethylhexyl Methoxycinnamate,
Octocrylene,
Phenylbenzimidazole Sulfonic Acid,
Benzophenone-3,
Benzophenone-4,
Benzophenone-5,
4-Methylbenzylidene Camphor,
Terephthalylidene Dicamphor Sulfonic Acid,
Disodium Phenyl Dibenzimidazole Tetrasulfonate,
2,4,6-Tris(diisobutyl 4′-aminobenzalmalonate)-s-triazine,
Anisotriazine,
Ethylhexyl Triazone,
Diethylhexyl Butamido Triazone,
Methylene Bis-Benzotriazolyl Tetramethylbutylphenol,
Drometrizole Trisiloxane,
Polysilicone-15,
1,1-Dicarboxy (2,2′-dimethylpropyl)-4,4-diphenylbutadiene,
2,4-Bis[5-1(dimethylpropyl)benzoxazol-2-yl-(4-phenyl)imino]-6-(2-ethylhexyl)imino-1,3,5-triazine, and their mixtures.

The inorganic protoprotective agents are preferably chosen from pigments or alternatively nanopigments (mean size of the primary particles: generally between 5 nm and 100 nm, preferably between 10 nm and 50 nm) formed of metal oxides which may or may not be coated, such as, for example, nanopigments formed of titanium oxide (amorphous or crystalline in the rutile and/or anatase form), iron oxide, zinc oxide, zirconium oxide or cerium oxide, which are all UV photoprotective agents well known per se. Furthermore, conventional coating agents are alumina and/or aluminium stearate. Such nanopigments formed of metal oxides, which may or may not be coated, are disclosed in particular in Patent Applications EP 518 772 and EP 518 773.

The photoprotective agents can be present in the fourth composition according to the invention in, for example, proportions ranging from 0.1 to 20% by weight, with respect to the total weight of the composition, and preferably ranging from 0.2 to 15% by weight, with respect to the total weight of the composition.

The compositions used in the process according to the invention can be placed in a packaging or kit.

Another subject-matter of the present invention is a kit comprising:
a first packaging comprising a composition which comprises at least 5% by weight of metal oxide particles,
at least one second packaging comprising a peeling composition which comprises at least 5% by weight of at least one desquamating agent,
a third packaging comprising a composition comprising a thermal or mineral water having a mineral content of at least 300 mg/l,
a fourth packaging comprising a composition which comprises at least one anti-ageing active principle chosen from: compounds which enhance the synthesis of collagen and/or of elastin and/or of glycosaminoglycans and/or of proteoglycans and/or of fibronectin and/or of laminin, compounds which inhibit the decomposition of collagen and/or of elastin, skin relaxants, agents which inhibit the glycation of proteins, agents which enhance the proliferation of keratinocytes and/or of fibroblasts, agents which enhance the differentiation of keratinocytes, and their mixtures.

The terms “first”, “second”, “third” and “fourth” packagings used above do not have the effect of differentiating the packagings with regard to their appearance but with regard to the compositions present therein. Some or all may thus be provided in the same physical form.

However, according to a preferred embodiment of the invention, the first packaging can be provided in the form of a tube, preferably having a capacity ranging from 30 to 40 ml.

The second packaging can be a sachet comprising a wipe on which the second composition is impregnated. The kit according to the invention can, for example, comprise three sachets of this type.

The third packaging can be provided in the form of an aerosol device comprising the thermal or mineral water and a propellent gas which can be any liquefiable gas commonly used in aerosol devices, such as dimethyl ether, C₃₋₅ alkanes, 1,1-difluoroethane, mixtures of dimethyl ether and of C₃₋₅ alkanes, and mixtures of 1,1-difluoroethane and of dimethyl ether and/or of C₃₋₅ alkanes. Preference is given very particularly to the use as propellent agent of C₃₋₅ alkanes and in particular of propane, n-butane and isobutane. This device can thus have a capacity of 25 to 100 ml, advantageously of 50 ml, for example.

The fourth packaging can be provided in the form of a pump-action spray, tube or pot, for example, which can in particular have a capacity of 30 to 50 ml.

The kit described above can also comprise instructions including directions on its form of use, for the implementation of the method described above.

A preferred form of use, also denoted by the term “routine”, comprises:
the implementation of the method described above on the morning of a day of the week, and
the application to the skin of the fourth composition on the following six days,
   these two stages being repeated for three to four consecutive weeks.

The routine itself can be employed from one to four or more times annually, ideally in each season. It can be used to prepare the skin for the implementation of a dermatological peeling carried out by a practitioner or for maintaining the effects of such a peeling after it has been carried out.

Each of the two stages of this routine can be followed by the application of a makeup composition.

The invention will now be illustrated by the following nonlimiting examples. In these examples, the amounts are shown as percentage by weight.

### EXAMPLES

### Example 1

### Anti-Ageing Kit

A kit can be manufactured which comprises, in addition to an aerosol device comprising 50 ml of water from Vichy, a tube including the microdermabrasion composition A (first composition according to the invention), the peeling composition B (second composition according to the invention) and the composition C with an anti-ageing effect (fourth composition according to the invention) described below.

### Microdermabrasion Composition A (Cream)

An O/W emulsion having the composition below is prepared in a way conventional to a person skilled in the art:

| | | |
|---|---|---|
| Phase A | PEG (100 EO) stearate | 0.75% |
| | Glyceryl stearate | 0.75% |
| | Stearyl alcohol | 1.5% |
| | Mineral oil | 10% |
| | Apricot kernel oil | 2% |
| | Polydimethylsiloxane | 1% |
| Phase B | Demineralized water | q.s. for 100% |
| | 1% Polysaccharide* | 1% |
| | Polyacrylamide | 0.3% |
| | Butylene glycol | 3% |
| | Propylene glycol | 3% |
| Phase C | Aluminium oxide** | 15% |
| Phase D | Demineralized water | 5% |
| | Preservatives | q.s. |

*Fucogel 1000 PP from Solabia
**Dermagrain 900 from MarkeTech International

This cream can be applied in the morning to the wetted face, avoiding the outline of the eyes and lips, by massaging with the fingertips for approximately 1 minute 30, followed by rinsing with water and by drying using a clean towel. It makes it possible to improve the radiance of the complexion, to smooth its surface and to reduce the appearance of wrinkles. It prepares the skin for the application of the peeling composition.

### Peeling Composition B (Aqueous Gel)

A wipe is impregnated using the following composition:

| | |
|---|---|
| Glycolic acid | 9.5% |
| Lactic acid | 0.45% |
| Gelling agent | 0.25% |
| Ethanol | 5% |
| Neutralizing agent | 3.4% |
| Water | q.s. for 100% |

The wipe is left in contact with the skin for approximately 3 minutes. This composition makes it possible to close the pores, soften wrinkles and fine lines and accelerate skin replacement. This stage is followed by the application to the skin of a mineral water, such as water from the Vichy basin, for example in the form of a spray, and then by the drying of the skin using a clean towel, for the purpose of soothing the skin.

Composition C with an Anti-Ageing Effect (O/W Emulsion)

| | |
|---|---|
| Tocopherol | 0.025% |
| Arginine PCA | 0.1% |
| Glycerol | 7% |
| Surfactants | 2.5% |
| Oils | 8% |
| UV screening agents | 10% |
| Thickeners | 1% |
| Cetyl alcohol | 2% |
| Fatty acids | 1% |
| Myristyl myristate | 2% |
| Disodium EDTA | 0.1% |
| Preservatives | 1% |
| Ethanol | 4% |
| Neutralizing agent | q.s. pH 6 |
| Water | q.s. for 100% |

This composition makes it possible to smooth out wrinkles, to tone up the skin and to restore radiance thereto.

### Example 2

### Microdermabrasion Composition A′ (Foaming Gel)

The following composition is prepared in a way conventional to a person skilled in the art:

| Phase A | Demineralized water | q.s. for 100% |
|---|---|---|
| | EDTA | 0.2% |
| | Preservative | q.s. |
| | Acrylic copolymer (Pemulen TR1) | 1% |
| | Sodium lauryl sarcosinate | 2% |
| | Sodium laureth sulphate | 5% |
| Phase B | Demineralized water | 10% |
| | 1% Polysaccharide* | 2% |
| | Xanthan gum | 0.3% |
| | Butylene glycol | 3% |
| | Propylene glycol | 3% |
| | Preservative | q.s. |
| | Triethanolamine | 0.8% |
| | Magnesium oxide** | 6% |

*Fucogel 1000 PP from Solabia
**Magnaderm 100 from MarkeTech International

The invention method and kit is preferably used by subjects desirous of the benefits noted herein, subjects “in need of” these benefits, including softening cutaneous signs of ageing. Such subjects are typically suffering from cutaneous signs of ageing, such as by self diagnosis or cosmetician or medical diagnosis, or are at recognized and appreciated risk of developing such conditions and who use the invention methods and kit to combat these effects. In this regard, the invention process can be viewed as one for delaying the onset of the appearance of, and/or for reducing signs of, ageing of the skin.

Naturally, one using the invention as disclosed will preferably use an amount of the invention compositions effective to reduce or soften signs of cutaneous ageing. Such amounts are inclusive of an amount of the compositions described herein at the disclosed concentrations of active ingredients sufficient to cover the area of the skin being treated in a single application, and of course includes that amount applied upon repeated application, for example on a daily basis over a course of days, weeks, etc., for example according to the routine described above. In a preferred embodiment the invention process includes multiple applications of the invention composition to the area(s) of skin in need of attention.

The above written description of the invention provides a manner and process of making and using it such that any person skilled in this art is enabled to make and use the same, this enablement being provided in particular for the subject matter of the appended claims, which make up a part of the original description and including a method for caring for the skin, intended to soften cutaneous signs of ageing, comprising the successive application, to an area of skin wetted beforehand:
of a microdermabrasion composition including at least 5% by weight of metal oxide particles, which is applied by massaging, and then removed by washing,
of a peeling composition including at least 3% by weight of at least one desquamating agent,
of a soothing composition comprising a thermal or mineral water having a mineral content of at least 300 mg/l,
of an anti-ageing composition including at least one anti-ageing active principle chosen from: compounds which enhance the synthesis of collagen and/or of elastin and/or of glycosaminoglycans and/or of proteoglycans and/or of fibronectin and/or of laminin; compounds which inhibit the decomposition of collagen and/or of elastin; skin relaxants; agents which inhibit the glycation of proteins; agents which enhance the proliferation of keratinocytes and/or of fibroblasts; agents which enhance the differentiation of keratinocytes; and their mixtures.

Another preferred embodiment of the invention similarly fully described and enabled is a kit including the four compositions described above separated from one another.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out. Terms such as “contain(s)” and the like as used herein are open terms meaning ‘including at least’ unless otherwise specifically noted.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A kit for skin care intended to soften cutaneous signs of ageing, comprising:
a first packaging comprising a microdermabrasion composition which comprises at least 5% by weight of metal oxide particles,
at least one second packaging comprising a peeling composition which comprises at least 5% by weight of at least one desquamating agent,
a third packaging comprising a soothing composition comprising a thermal or mineral water having a mineral content of at least 300 mg/l,
a fourth packaging comprising a composition which comprises at least one anti-ageing active principle chosen from: compounds which enhance the synthesis of collagen and/or of elastin and/or of glycosaminoglycans and/or of proteoglycans and/or of fibronectin and/or of laminin, compounds which inhibit the decomposition of collagen and/or of elastin, skin relaxants, agents which inhibit the glycation of proteins, agents which enhance the proliferation of keratinocytes and/or of fibroblasts, agents which enhance the differentiation of keratinocytes, and their mixtures.

2. The kit according to claim 1, wherein the metal oxide particles comprise at least one selected from the group consisting of magnesium oxide particles and aluminium oxide particles.

3. The kit according to claim 1, wherein the metal oxide particles have a mean particle size ranging from 100 to 180 μm.

4. The kit according to claim 1, wherein the metal oxide particles represent from 10 to 30% of the total weight of the microdermabrasion composition.

5. The kit according to claim 1, wherein the microdermabrasion composition has a pH of between 5.5 and 7.

6. The kit according to claim 1, wherein the microdermabrasion composition further comprises at least one heterogeneous polysaccharide.

7. The kit according to claim 1, wherein the peeling composition comprises an α-hydroxy acid as a desquamating agent.

8. The kit according to claim 1, wherein the peeling composition has a pH of between 3.5 and 4.5.

9. The kit according to claim 1, wherein the peeling composition is impregnated on a wipe.

10. The kit according to claim 1, wherein the soothing composition comprises 100% by weight of mineral or thermal water.

11. The kit according to claim 1, wherein the anti-ageing active principle is ascorbic acid.

12. The kit according to claim 1, wherein the anti-ageing composition has a pH of between 5.5 and 6.5.

13. The kit according to claim 1, wherein the microdermabrasion composition and/or the anti-ageing composition are in the form of an oil-in-water emulsion.

14. The kit according to claim 1, wherein the peeling composition is in the form of an aqueous gel.

15. The kit according to claim 1, wherein the anti-ageing composition further comprises at least one chosen from the group consisting of at least one moisturizing agent, at least one antioxidant or agent for combating free radicals, at least one organic or inorganic photoprotective agent which is active in the UV-A and/or UV-B region, and mixtures thereof.

16. The kit according to claim 1, wherein the peeling composition comprises glycolic acid, as a desquamating agent.

17. The kit according to claim 1, wherein the anti-ageing composition comprises glycerol.
